(19) United States
(12) Patent Application Publication
(10) Pub. No.: US 2024/0341657 A1
(43) Pub. Date: Oct. 17, 2024

(54) SYSTEMS AND METHODS FOR ADAPTING ECG DATA RATE BASED ON GRADIENT ACTIVITY

(71) Applicant: KONINKLIJKE PHILIPS N.V., EINDHOVEN (NL)

(72) Inventor: PAUL FRANZ REDDER, NEWBERRY, FL (US)

(21) Appl. No.: 18/681,537

(22) PCT Filed: Aug. 8, 2022
(86) PCT No.: PCT/EP2022/072182

§ 371 Date: Feb. 6, 2024

Related U.S. Application Data
(60) Provisional application No. 63/231,284, filed on Aug. 10, 2021.

(51) Int. Cl. A61B 5/33; A61B 5/00; A61B 5/055

(52) U.S. Cl. CPC A61B 5/33; A61B 5/0002; A61B 5/055; A61B 5/7285

(57) ABSTRACT

A system (400) for monitoring a physiological parameter of a patient including a wireless signal unit (60, 500) having a transceiver (515) configured to transmit, by a communication link (CL), wireless data associated with the physiological parameter of the patient at a default transfer rate; a patient monitor (70) configured to receive the wireless data transmitted from the transceiver at the default transfer rate; and a processor (64, 505, 535) communicably coupled with the wireless signal unit. The processor is configured to: (i) receive and preprocess (650) an input signal comprising a signal corresponding to the physiological parameter of the patient and transient gradient signals from a gradient system; (ii) determine (650) a gradient signal activity value of the gradient system; and (iii) dynamically adjust (670) the default transfer rate of the wireless data transmitted from the transceiver to an adjusted transfer rate based on the gradient signal activity value.

### FIELD OF THE DISCLOSURE

The present disclosure is directed generally to physiological signal monitoring in shielded environments. It finds particular application in conjunction with electrocardiography (ECG) in magnetic resonance imaging, and will be described with particular reference thereto. However, it will be understood that it also finds application in other usage scenarios and is not necessarily limited to the aforementioned application. More specifically, the present disclosure is directed to systems and methods for wirelessly monitoring medical and health data (e.g., vital signs and/or electrocardiogram data) in shielded areas, such as magnetic resonance (MR) exam rooms, by reducing a data rate based on gradient signal activity using digital signal processing resources.

### BACKGROUND

Magnetic resonance imaging (MRI) systems employ high power gradient systems to phase encode the imaging data during acquisition. Unfortunately, these high power gradient signals couple to the ECG leads used for patient monitoring during the MRI image acquisition and cause gradient induced artifacts. These gradient signals are driving the need for high dynamic range receivers and increasing the effective number of bits (ENOB) of the received signal, which results in a larger data rate required to communicate the ECG data to a patient monitor. The larger data rate link is needed to communicate the ECG data while preserving the ECG information content. The system dynamic range has to accommodate the small desired ECG signal along with the high dynamic range interferer (e.g., the high power gradient signal). Compounding this situation is that the frequency content of the gradient signals are located at or very close to the desired ECG signal. Traditional data encoding techniques cannot be used without adversely affecting the desired ECG signal.

The high dynamic range receiver along with the high data rate communication links require additional power, thereby increasing cost. For battery-powered ECG sensors, this increased power consumption drives the need for more charging cycles or battery replacement. The additional power consumption further reduces the through-put of the MRI system unless costlier measures are implemented, i.e., incorporating additional sensors, chargers, or batteries.

Thus, there is a need in the art for improved apparatus and systems for wirelessly monitoring medical and health data in shielded areas such that they are less expensive and more efficient.

### SUMMARY OF THE DISCLOSURE

The present disclosure is directed generally to inventive apparatus and systems for wirelessly monitoring one or more physiological signals of a patient in a shielded environment, such as a magnetic resonance environment, or any environment that includes intermittent distortion of the one or more physiological signals of the patient. Various embodiments and implementations herein are directed to improved systems or apparatus that utilize digital signal processing to measure the signal dynamic range of the system during periods of time when transient gradient signals are present and when the transient gradient signals are not present. An algorithm uses the values of the signal dynamic range during the periods of time to adjust a data link rate during MRI image acquisition. The data link rate can be increased or maximized when the transient gradient signals are present and decreased or reduced when the transient gradient signals are not present during MRI image acquisition. The shielded environment can comprise a shielded magnet room such as an MR room having shielding walls that comprise steel or cooper or any suitable alternative. The algorithm is provided in or near a wireless ECG signal unit that is positionable inside a bore of a magnet during image acquisition. The wireless ECG signal unit wirelessly communicates signals corresponding to a physiological parameter of a patient to a base unit or patient monitor outside the bore of the MRI magnet yet within the shielded room based on the dynamically adjustable transfer rate. Applicant has recognized and appreciated that the high dynamic range receiver of the wireless ECG signal unit and the high data rate communication links are not needed at all times during MRI image acquisition, particularly when transient gradient signals are not present. Applicant has further recognized and appreciated that the power consumption of the monitoring systems can be significantly reduced by dynamically adjusting the transfer rate of the wireless data transmission based on the transient gradient signal activity.

Generally, in one aspect, a wireless signal unit is provided. The wireless signal unit includes a transceiver configured to transmit, by a communication link, wireless data associated with a physiological parameter of a patient at a default data transfer rate within a patient information system and a processor. The processor is configured to receive and preprocess an input signal comprising a signal corresponding to the physiological parameter of the patient and transient gradient signals from a gradient system; determine a gradient signal activity value of the gradient system; and dynamically adjust the default transfer rate of the wireless data transmitted from the transceiver to an adjusted data transfer rate based on the gradient signal activity value.

In an embodiment, the gradient system is part of a magnetic resonance imaging (MRI) system.

In an embodiment, the signal corresponding to the physiological parameter of the patient comprises an ECG signal.

In an embodiment, the adjusted data transfer rate comprises a minimum rate needed to communicate the signal corresponding to the physiological parameter taking into consideration a data phase encoding process occurring during an image acquisition process.

In an embodiment, the transceiver is configured to transmit the wireless data associated with the input signal to a patient monitor comprising a display, wherein the patient monitor is part of the patient information system or otherwise connected to the patient information system.

In an embodiment, the processor is further configured to determine an effective number of bits of the received input signal.

In an embodiment, the processor is further configured to compare the determined effective number of bits of the received input signal with a predetermined or default maximum effective number of bits used when the transient gradient signals are present.

In an embodiment, the processor is further configured to reduce the default data transfer rate when the determined effective number of bits of the received input signal is lower than the predetermined or default maximum effective number of bits used when the transient gradient signals are present.

In an embodiment, the processor is further configured to reduce the default transfer rate according to a predetermined reduced transfer rate associated with the determined effective number of bits of the received input signal.

Generally, in another aspect, a system for monitoring a physiological parameter of a patient is provided. The system includes a wireless signal unit comprising a transceiver configured to transmit, by a communication link, wireless data associated with the physiological parameter of the patient at a default data transfer rate. The system further includes a patient monitor comprising a receiver configured to receive the wireless data transmitted from the transceiver of the wireless transceiver at the default data transfer rate and a processor. The processor of the system is communicably coupled with the wireless signal unit and configured to (i) receive and preprocess an input signal comprising a signal corresponding to the physiological parameter of the patient and transient gradient signals from a gradient system; (ii) determine a gradient signal activity value of the gradient system; and (iii) dynamically adjust the default data transfer rate of the wireless data transmitted from the transceiver to an adjusted transfer rate based on the gradient signal activity value.

In an embodiment, the gradient system is part of a magnetic resonance imaging (MRI) system.

In an embodiment, the signal corresponding to the physiological parameter of the patient comprises an ECG signal.

In an embodiment, the adjusted data transfer rate comprises a minimum rate needed to communicate the signal corresponding to the physiological parameter taking into consideration a data phase encoding process occurring during an image acquisition process.

In an embodiment, the patient monitor further includes a monitor processor configured to post-process the wireless data received from the transceiver; and a display configured to display the post-processed wireless data.

In an embodiment, the processor is further configured to: (i) determine an effective number of bits of the received input signal; (ii) compare the determined effective number of bits of the received input signal with a predetermined or default maximum effective number of bits used when the transient gradient signals are present; and (iii) reduce the default data transfer rate when the determined effective number of bits of the received input signal is lower than the predetermined or default maximum effective number of bits used when the transient gradient signals are present.

In various implementations, a processor or controller may be associated with one or more storage media (generically referred to herein as “memory,” e.g., volatile, and non-volatile computer memory such as RAM, PROM, EPROM, and EEPROM, floppy disks, compact disks, optical disks, magnetic tape, etc.). In some implementations, the storage media may be encoded with one or more programs that, when executed on one or more processors and/or controllers, perform at least some of the functions discussed herein. Various storage media may be fixed within a processor or controller or may be transportable, such that the one or more programs stored thereon can be loaded into a processor or controller so as to implement various aspects as discussed herein. The terms “program” or “computer program” are used herein in a generic sense to refer to any type of computer code (e.g., software or microcode) that can be employed to program one or more processors or controllers.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the various embodiments.

FIG. 1 is a schematic representation of an example set of cardiovascular (cv) electrode patches, according to aspects of the present disclosure.

FIG. 2 is a schematic representation of an example standard cv electrode patch, according to aspects of the present disclosure.

FIG. 3 diagrammatically illustrates an exemplary placement of a set of cv electrode patches, according to aspects of the present disclosure.

FIG. 4 shows an embodiment of one or more electrode patches with a parallel lead processing system in a magnetic resonance (MR) environment, according to aspects of the present disclosure.

FIG. 5 schematically depicts a wireless signal unit of a lead processing system, according to aspects of the present disclosure.

FIG. 6 is a flowchart of a method for dynamically adjusting a transfer rate of wireless data from a wireless transceiver, according to aspects of the present disclosure.

### DETAILED DESCRIPTION OF EMBODIMENTS

The present disclosure describes various embodiments of improved systems and apparatus for dynamically adjusting a wireless data transfer rate between a wireless ECG signal unit that transmits patient data and a patient monitor unit that receives the transmitted patient data from the ECG signal unit. Applicant has recognized and appreciated that the high dynamic range receiver of the wireless ECG signal unit and the high data rate communication links are not needed at all times during MRI image acquisition, particularly when transient gradient signals are not present. Applicant has further recognized and appreciated that the power consumption of the monitoring systems can be significantly reduced by dynamically adjusting the transfer rate of the wireless data transmission based on the transient gradient signal activity. The improved apparatus and systems include a wireless signal unit comprising a transceiver configured to transmit, by a communication link to a patient information system, wireless data associated with a physiological parameter of a patient; and a processor in communication with the wireless signal unit. The processor is configured to preprocess an input signal comprising a signal corresponding to the physiological parameter of the patient and transient gradient signals from a gradient system. The processor is further configured to: (i) determine a gradient signal activity value of the gradient system; and (ii) dynamically adjust a transfer rate of the wireless data transmitted from the transceiver based on the gradient signal activity value. The improved apparatus and systems provide the advantages described herein as well as others as should be appreciated.

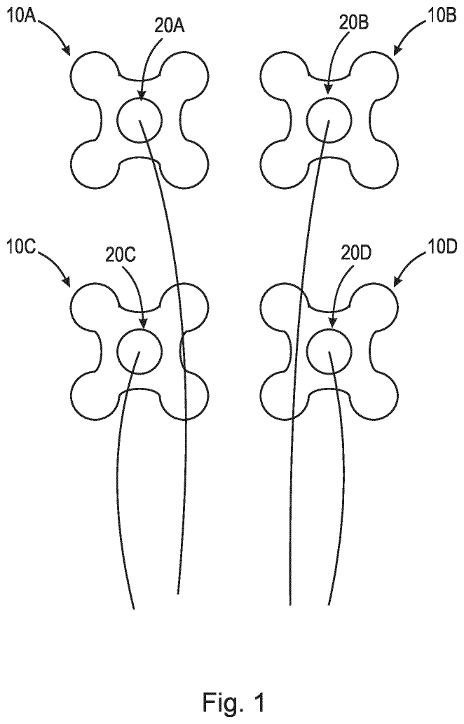

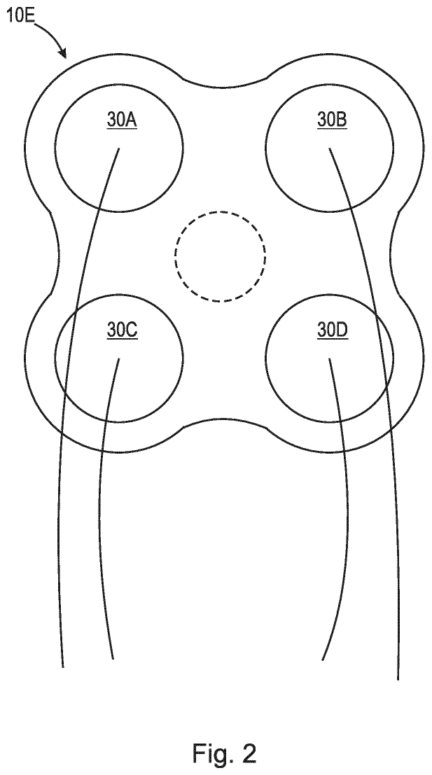

Referring to FIG. 1, a schematic representation of an example set cardiovascular (cv) electrode patches is provided. FIG. 2 shows a schematic representation of an example standard electrode patch. The following should be appreciated in view of FIGS. 1 and 2. Electrode patches 10A, 10B, 10C, and 10D of FIG. 1 are typically provided for female and/or overweight patients in the example configuration. Patch 10E of FIG. 2 is typically provided for those patients that do not necessitate the configuration shown in FIG. 1. Each of patches 10A-10E includes a flexible patch material or substrate made of plastic or polymer layers, and are configured to be attached to the skin of a patient. An adhesive coating layer can be provided for attachment. Each of patches 10A-10D in FIG. 1 includes an electrode 20A-20D, respectively, while patch 10E includes four electrodes 30A-30D within the same patch. Each electrode of electrodes 20A-20D is attached to or formed in or on a center of a patch of patches 10A-10D, respectively. Electrodes 30A-30D are attached to or formed in or on each rounded corner of patch 10E. For example, electrode 30A is arranged or located in or on an upper left rounded corner of patch 10E, electrode 30B is arranged or located in or on an upper right rounded corner of patch 10E, electrode 30C is arranged or located in or on a lower left rounded corner of patch 10E, and electrode 30D is arranged or located in or on a lower right rounded corner of patch 10E. Any suitable configurations are contemplated and the present disclosure should not be limited by the configurations depicted in FIGS. 1 and 2.

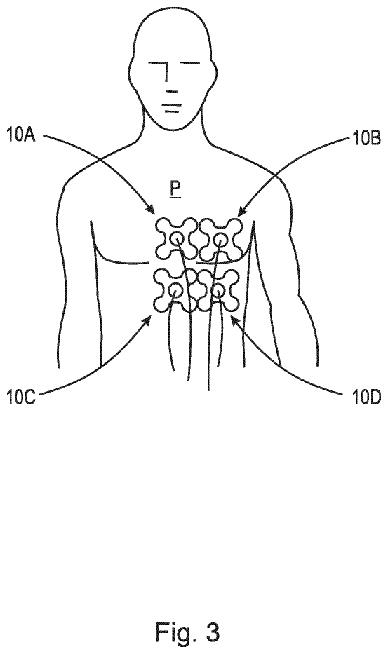

Electrodes 20A-20D and 30A-30D of FIGS. 1 and 2 are configured to sense electrical activity of a patient's heart. The electrodes can include Ag-AgCL based electrodes or any suitable alternatives. As shown in FIG. 3, patches 10A-10D and the corresponding electrodes 20A-20D of FIG. 1 can be attached to a patient P in a suitable configuration to sense electrical activity of the patient's heart. Although not shown, patch 10E and the corresponding electrodes 30A-30D of FIG. 2 can also be attached to patient P in a similar suitable configuration to sense electrical activity of the patient's heart. In embodiments, patch 10E can include an opening in the center, for example, for neonatal patients.

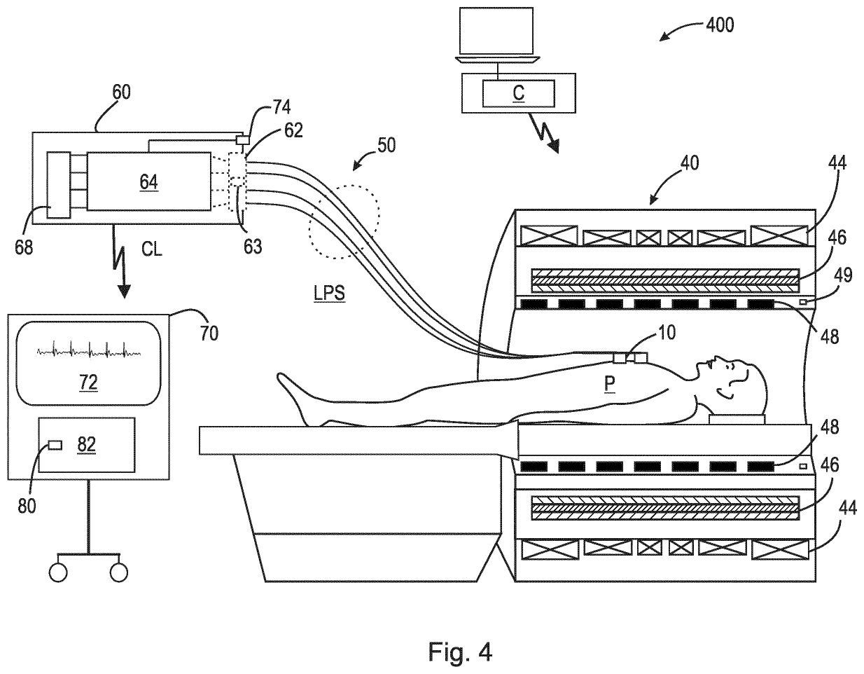

Referring to FIG. 4, an embodiment of one or more electrode patches with a lead processing system (LPS) in a magnetic resonance (MR) environment is depicted. FIG. 4 shows a monitoring system 400 including a partially cutaway side view of a MRI scanner 40. The system 400 broadly comprises MR scanner 40, one or more controllers C, wireless signal unit 60, and patient monitor 70. System 400 also comprises a patient support to support patient P for scanning and/or to position patient P in a desired position and/or orientation in relation to the scanner 40. In embodiments, the positioning of patient P may be partially or entirely controlled by controllers C. As further described herein, wireless signal unit 60 is configured to generate a waveform signal from the detected electrical activity and wirelessly transmit the waveform signal to patient monitor 70 for display. Since the wires from the electrodes are electrically conductive, the lead wires and other wires are susceptible to interference currents induced by the gradient field pulses applied to the patient P during image acquisition. The induced currents in the electrodes and/or the electrode wires produce gradient noise interference on the ECG signal. The interference from gradient pulses typically has an amplitude of about ±100 mV, but can reach as high as ±5 V. An ECG typically has an amplitude of about 300 uV to 5 mV peak-to-peak. To handle the ECG signal with gradient noise interference, an analog-to-digital converter having a high dynamic range is necessary. The dynamic range refers to the ratio between the maximum input signal and intrinsic noise level and can be expressed as an effective number of bits (ENOB). In embodiments, an ADC with a high dynamic range (e.g., many effective bits) such as 16-bits is used to accommodate the small desired ECG signal along with the high dynamic range interferer (e.g., the high power gradient signal). However, using a high number of bits continuously requires a lot of power consumption and cost. The improved apparatus and systems of the present application are configured to optimize the effective number of bits of the received signal, and thereby the data rate, needed to communicate the minimum data rate without any impact on the desired ECG signal due to data encoding.

Scanner 40 may include a bore situated between opposing ends of the scanner. In embodiments, the opposing ends of the scanner 40 are open. Scanner 40 further includes a housing which comprises main magnet 44, gradient coils 46, and radio-frequency (RF) coils 48. At least a portion of patient P can be located in an examining region of MR scanner 40, such as a horizontal bore, vertical bore, c-type, and the like. Scanner 40 can further include a cooling mechanism (e.g., a cryogenic cooling system). For example, magnet 44 can include a plurality of magnets or superconducting coils disposed inside a cryoshrouding. There are a number of different MRI scanner systems with different configurations, i.e., panoramic systems. It should be appreciated that the invention should not be limited in any way by the configuration depicted.

Main magnet 44 is configured to generate a static magnetic field directed generally parallel to a cylinder axis of the bore of the scanner 40. Resistive main magnets can also be used in embodiments. The main magnet 44 may be an annular (e.g., ring-shaped) magnet. However, in other embodiments, the main magnet 44 may include any suitable magnet or magnets such as an annular magnet, a planar magnet, a split magnet, an open magnet, a semi-circular magnet, etc. The main magnet 44 can be made of any suitable material such as a superconducting material and/or may operate under the control of the controller C.

Gradient coils 46 can comprise x-, y-, and z-gradient coils that are configured to generate gradient magnetic fields along one or more corresponding axes under control of the controller C. The gradient magnetic fields manipulate and encode magnetic resonance in tissues of the patient P. The coils 46 can selectively produce magnetic field gradients in the bore of the housing of the scanner 40 by the controller C.

Each of the one or more radio frequency (RF) coils 48 can be embodied as a body coil, a head coil, a surface coil, or another local coil for selectively exciting magnetic resonances. The one or more RF coils 48 can be configured as transmission coils or an array of coils to generate RF excitation pulses to induce magnetic resonance in tissues of the patient P. The RF coils 48 can be positioned within the bore of the scanner 40 to obtain images of a desired scanning volume within the bore. The controller C can control the coils 48. In embodiments, scanner 40 further includes a RF transmitter 49 coupled to the one or more RF coils to selectively inject RF excitation pules into the patient to be imaged.

By selectively operating the magnetic field gradient coils 46 and the RF coils 48, a magnetic resonance may be generated and spatially encoded in at least a portion of a region of interest of the imaging patient P. By applying selected magnetic field gradients via the gradient coils 46, a selected k-space trajectory can be traversed, such as a Cartesian trajectory, a plurality of radial trajectories, a spiral trajectory and so forth.

The one or more controllers C can control the overall operation of the system 400 and may include one or more logic devices such as processors (e.g., micro-processors, etc.). Controllers C can include one or more of a main magnet controller, a gradient controller, and an RF controller. The main magnet controller can control the operation of the main magnet 44. The gradient controller can control the operation of the gradient coils 46. The RF controller can control the operation of the RF coils 48. The one or more controllers C can include at least one controller such as a system controller and may be formed integrally with, or separately from, scanner 40. For example, the controller may be remotely located from the scanner 40 and communicate with one or more components of the system including the main magnet, gradient coils, and the RF coils via wired and/or wireless communication methods. Further, the controller may communicate with one or more of the above elements via one or more networks (e.g., a wide area network (WAN), a local area network (LAN), the Internet, a proprietary communication bus, a controller area network (CAN), a telephone network, etc.). It should also be appreciated that the one or more controllers C can include at least one controller such as a system controller and may be formed integrally with, or separately from, patient monitor 70 or any other component of system 400.

The electrodes in the attached patch 10 sense electrical activity of the heart of patient P. Such electrical activity is transmitted from the electrodes to the cables 50 and to wireless signal unit 60 or an ECG monitoring device or ECG unit. The terms “unit” and “device” are used interchangeably herein to refer to elements 60 and 500. It should be appreciated that ECG monitoring device 60 is preferably a handheld, battery-powered device that is configured to be in close proximity to the electrodes on the patient P and within or outside of the bore during MRI image acquisition. In FIG. 4, device 60 is shown a distance from patient P for illustration purposes only. In embodiments, device 60 includes a chargeable accumulator or battery. As used herein, MRI image acquisition refers to the reconstruction of two or three dimensional visualization of anatomic data contained within the magnetic resonance imaging data. The visualization can be performed using a computer.

In embodiments, the cables 50 transmit the ECG data stream directly to processor 64 so that the gradient activity can be extracted from the data stream such as by processor 505 and/or DSP 535 discussed below. In embodiments where the ECG data stream proceeds directly to processor 64, the processor can include a signal transition stage to convert the data stream from analog-to-digital before proceeding to additional processing steps. It should also be understood that the conversion from analog-to-digital during a signal transition stage can be carried out prior to being received at processor 64. In alternate embodiments, cables 50 can transmit the ECG data stream to a separate gradient receiver system, such as, circuit or receiver 62 in ECG device 60. In such embodiments, the receiver 62 converts the currents from the electrodes to ECG lead signals. In other words, receiver 62 generates a waveform signal. Receiver 62 can comprise a RF reception or receiver coil such as a wireless MRI coil or an array of similar local coils. Receiver 62 can further include a RF reception antenna 63 that can be tuned to a desired frequency of interest, i.e., the gradient signals generated by the MR system, and have a corresponding suitable bandwidth. In embodiments, antenna 63 can also be used for wireless communications with controller C. In embodiments, device 60 can comprise a separate antenna for wireless communication with the controller C.

In embodiments, the components of system 400, including ECG sensors, device 60, patient monitor 70, and controllers C, communicate wirelessly via a gateway that connects to a healthcare facility's network and/or transmits data or information to data storage for monitoring, control, or evaluating in real-time or offline after storage. Wireless sensors are also beneficial because they avoid using conductive wires that could otherwise couple with magnetic field gradients and heat up due to RF heating if improperly routed during the MR procedure.

Receiver 62 can comprise or be coupled to a variety of additional circuitry elements as described or otherwise contemplated herein. However, the present invention is not to be limited by the following illustrative examples. It should be appreciated that the additional circuitry elements described or otherwise envisioned herein can additionally or alternatively be integrated within processor 64 and/or patient monitor 70. In embodiments, one or more additional circuitry elements are comprised within device 60 and one or more additional circuitry elements are comprised within patient monitor 70. In embodiments, the one or more additional circuitry elements comprised in device 60 are preprocessing elements and the one or more additional circuitry elements comprised in patient monitor 70 are post-processing elements.

Processor 64 of device 60 can be configured to receive and preprocess the ECG signal corresponding to the physiological parameter of the patient P and gradient signals. Any one or more of the circuitry elements described herein can be integrated within processor 64. In embodiments, the ECG signals can be conveyed in combination or separately and individually to a preamplifier to preamplify the analog ECG signal detected from the ECG electrodes. In embodiments, the preamplifier can be a high quality instrumentation amplifier which amplifies the differential signal present on the ECG signal electrodes. Embodiments of the system 400 can also include an inverter within processor 64 to invert the common-mode signal present on the signals. The system 400 can further include a high speed noise reduction filter or any suitable alternative to remove any unwanted frequencies and/or frequency components within processor 64. The filters, such as, a slew rate filter, a band pass filter, and/or a t-wave suppression filter, can filter the ECG lead signals in parallel and simultaneously. After the filtering steps, the system 400 can include an offset amplifier within processor 64 to add an offset voltage to ensure that the voltage is within the input signal specifications of an analog-to-digital converter (ADC) device 68. Thereafter, the complete waveform can be digitized by ADC device 68. It should be appreciated that ADC device 68 can be integrated within processor 64 in embodiments or separate from processor 64 in alternate embodiments. It should further be appreciated that device 60 can be a software-defined transceiver in embodiments such that components that have been traditionally implemented in hardware (e.g., filters, amplifiers, modulators/demodulators, detectors, etc.) are instead implemented by means of software.

In embodiments, processor 64 can further include additional circuitry elements to convert the signal to a wireless transmission medium such as a RF or infrared transmission medium or any other suitable medium. In embodiments, processor 64 includes an interference reduction circuit to receive the waveform signal from receiver 62 or otherwise and interference from the magnetic resonance gradient pulses. The circuit can output the physiological signal with reduced or removed magnetic interference. The reduced interference physiological waveform can be transmitted wirelessly to patient monitor 70 comprising display device 72 via communication link CL. Patient monitor 70 includes an antenna or receiver 80 to receive the physiological waveform from device 60. Patient monitor 70 can further include additional circuitry elements 82 to carry out post-processing of the received physiological waveform so that the waveform can be displayed on display 72.

Display device 72 can be configured to display a corresponding human readable ECG waveform after suitable processing. Examples of a display device 72 include a computer monitor, a television screen, a touch screen, Vector display, Flat panel display, Vacuum fluorescent display (VF), Light-emitting diode (LED) displays, Electroluminescent display (ELD), Plasma display panels (PDP), Liquid crystal display (LCD), Organic light-emitting diode displays (OLED), a projector, Head-mounted display, and the like. In a preferred embodiment, display 72 comprises an integral LED widescreen (touch screen) display to display the patient's vital signs at high resolution. In addition to the patient's vital signs, a battery level indicator of one or more batteries required for operating the monitor 70 and/or unit 60 can be displayed. Additionally, the display can further include an indicator representing the status of the communication link between the device 60 and the monitor 70. For example, an icon can be shown when the communication link has been established and a different icon can be shown when the communication link has yet to be established or has been disrupted. Patient monitor 70 further includes a power on/off button.

The processor 64 can receive MR cycle information from the scanner 40 and/or one or more controllers C via connection 74. In embodiments, connection 74 can be integrated within processor 64. In other embodiments, connection 74 can be separate from processor 64. MR cycle information provides a basis for the processor 64 to determine a gradient signal activity value of the system 400 and dynamically adjust a transfer rate of the wireless data transmitted to the patient monitor 70. In embodiments, a gradient signal activity value represents the presence of gradient signals or a lack of gradient signals. Thus, when a gradient signal activity value indicates that MR gradient signals are present, the processor 64 does not reduce the transfer rate of the communication link. When a gradient signal activity value indicates that MR gradient signals are not present, processor 64 can reduce the transfer rate of the communication link between device 60 and patient monitor 70.

In embodiments, the gradient signal activity value can represent an amount or size of the gradient signals. Thus, a gradient signal activity value can also represent if, and/or to what extent, the gradient signals meet or exceed a threshold value. Accordingly, if a gradient signal activity value indicates that MR gradient signals meet or exceed the threshold value, processor 64 allows the unit 60 to communicate with the patient monitor 70 at a default high data transfer rate. When the gradient signal activity value indicates that MR gradient signals fall below the threshold value, processor 64 reduces the rate at which the device 60 communicates with the patient monitor 70 from the default data transfer rate. The processor 64 can be configured to monitor the gradient signal activity value continuously during MRI image acquisition. Processor 64 can also derive a gradient signal activity value based on the timing of the applied MR sequences in embodiments or any other suitable process. The processor 64 can reduce the transfer rate of the wireless communication link to the minimum rate needed to transfer the ECG alone without the interference. In embodiments, the 16-bits can be reduced to 5 or 6 bits. In embodiments, when the transient gradient signals are not present processor 64 can determine the effective number of bits of the received signal without the interference from the gradient signals by determining the first non-zero number at the most significant bit. Any other suitable method of determining the effective number of bits of the received signal without the interference from the gradient signals is contemplated as well.

The various circuitry elements can be embodied in a programmed or configured microprocessor, a microcontroller, a graphic processing unit (GPU), an application-specific integrated circuit (ASIC), a field-programmable gate array (FPGA), and the like. For example, one or more microprocessors or processors can be configured to filter a plurality of ECG lead signals, detect the ECG lead signal on the filtered signals, detect the gradient signal activity level, determine the effective number of bits of the signal without the transient gradient signals; and adjust a transfer rate of the wireless data transmitted from the transceiver based on the detected gradient signal activity level value. The processors can output the processed lead signals to patient monitor 70 via the default transfer rate or one or more adjusted transfer rates.

The memory and storage described herein may be considered to be non-transitory machine-readable media. As used herein, the term non-transitory means to exclude transitory signals but to include all forms of storage, including both volatile and non-volatile memories. The disclosed filtering, detection, computation, and selection techniques are suitably implemented using a non-transitory storage medium storing instructions (e.g., software) readable and executable by the electronic data processing devices to perform the disclosed filtering, detection, computation, and selection techniques.

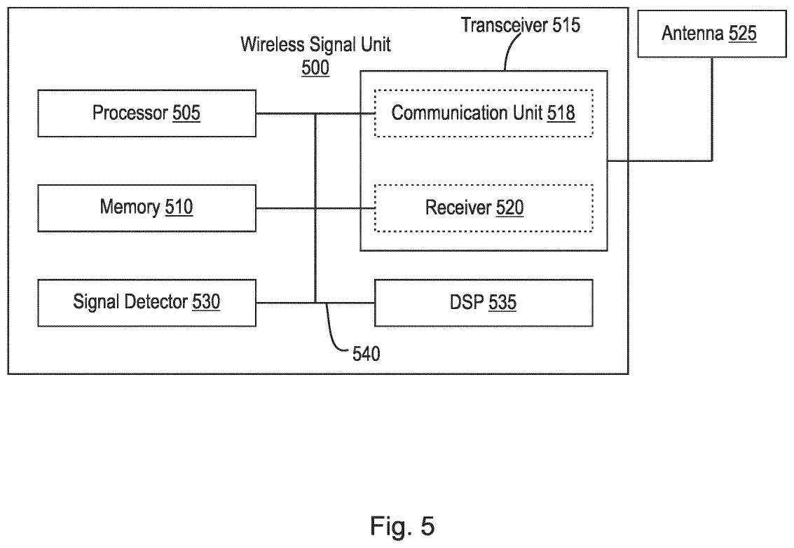

FIG. 5 shows a schematic representation of various components that may be utilized in a wireless signal unit such as device 60 described herein. The wireless signal unit of FIG. 5 may be used with system 400 and may be configured to implement the various methods described herein. Wireless signal unit 500 comprises processor 505, memory 510, transceiver 515, antenna 525, signal detector 530, and digital signal processor (DSP) 535. In embodiments, processor 505 and/or DSP 535 is used to extract the gradient signal activity from the ECG data stream from the electrodes. In alternate embodiments, device 60 can comprise or be in communication with a separate gradient signal receiver system as described above.

Processor 505 controls operation of unit 500 based on program instructions stored within memory 510. Memory 510 can comprise read-only memory (ROM) and/or random access memory (RAM) and/or non-volatile random access memory (NVRAM) and provides instructions and data to processor 505. Memory 510 can further include various memories such as, for example, L1, L2, or L3 cache or system memory. As such, the memory 510 may include static random access memory (SRAM), dynamic RAM (DRAM), flash memory, read only memory (ROM), or other similar memory devices. Memory 510 can store, among other things, an operating system. The RAM can be used by the processor for the temporary storage of data from the electrodes. It should be appreciated that various information described as being stored in memory 510 may be additionally or alternatively stored in a separate memory device. According to an embodiment, an operating system may contain code which, when executed by processor 505, controls the operation of one or more components of wireless monitoring system 400. Processor 505 performs logical and arithmetic operations based on the program instructions stored within memory 510. The instructions may be executable to implement the methods described herein. It will be apparent that, in embodiments where the processor implements one or more of the functions described herein in hardware, the software described as corresponding to such functionality in other embodiments may be omitted.

Processor 505 may comprise or be a component of a processing system with one or more processors. The one or more processors may be implemented with any combination of microprocessors, microcontrollers, multiple microcontrollers, circuitry, digital signal processors (DSPs) 535, field programmable gate array (FPGAs), application-specific integrated circuits (ASICs), a single processor, plural processors, programmable logic devices (PLDs), controllers, state machines, gated logic, discrete hardware components, dedicated hardware finite state machines, or any other suitable components that can perform calculations or other manipulations of information. The processing system can also include machine-readable media for storing software. Software shall be construed broadly to mean any type of instructions when referred to as software, firmware, middleware, microcode, hardware description language, or otherwise. The instructions, when executed by the one or more processors 505, cause the processing system to perform the various functions described herein.

While device 60, 500 and/or system 400 are generally shown as including one of each described component, the various components may be duplicated in various embodiments. For example, processor 505 may include multiple microprocessors that are configured to independently execute the methods described herein or are configured to perform steps or subroutines of the methods described herein such that the multiple processors cooperate to achieve the functionality described herein. Further, where one or more components is implemented in a cloud computing system, the various hardware components may belong to separate physical systems. For example, processors 505 and/or 535 may include a first processor in a first server and a second processor in a second server. Many other variations and configurations are possible.

Unit 500 further comprises transceiver 515 electrically coupled to antenna 525 to allow transmission and reception of data between unit 500 and patient monitor 70 described herein. In embodiments, transceiver 515 can be divided into communication unit 518 and receiver 520 and each are electrically coupled to antenna 525. Antenna 525 may be integrated within unit 500 or otherwise coupled or attached to unit 500. Transceiver 515 and/or communication unit 518 may include one or more devices for enabling communication with other hardware devices. For example, transceiver 515 and/or communication unit 518 may include a network interface card (NIC) configured to communicate according to the Ethernet protocol. Alternatively, transceiver 515 and/or communication unit 518 may implement a TCP/IP stack for communicating according to TCP/IP protocols. Various alternative or additional hardware or configurations for transceiver 515 and/or communication unit 518 are contemplated.

Unit 500 can also include a signal detector 530 and digital signal processor 535. Signal detector 530 can be configured to detect and quantify the signals received from the electrodes. Digital signal processor 535 can be configured to process the signals as described herein. The various components of wireless unit 500 can be coupled together by system bus 540 that can comprise a power bus, a control signal bus, and a status signal bus in addition to a data bus.

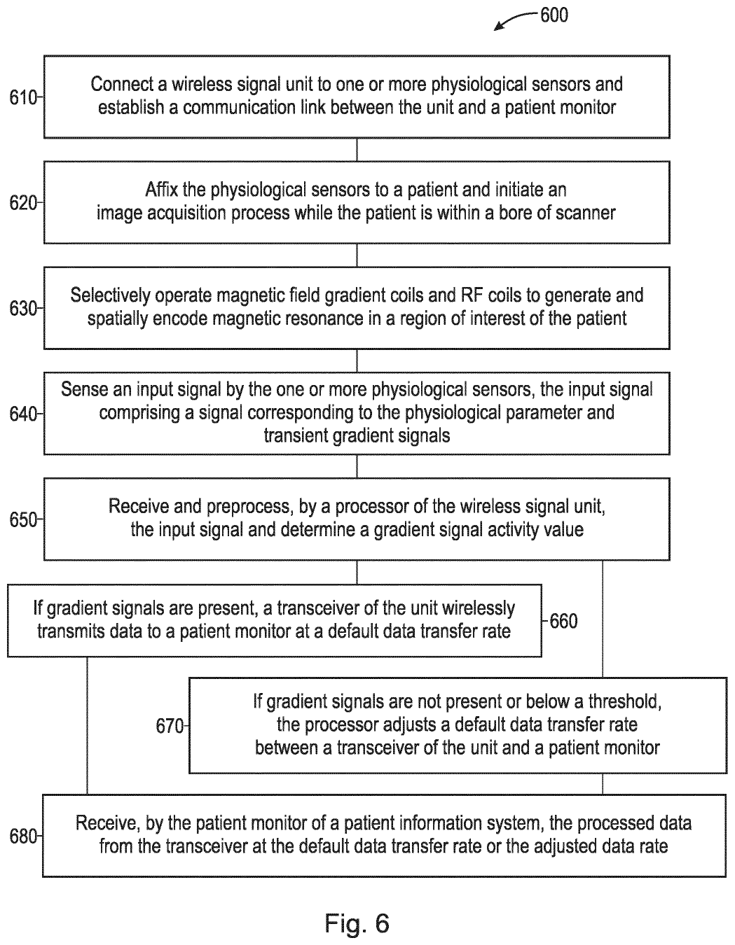

With reference to FIG. 6, an embodiment of a method 600 of using wireless unit 500 or device 60 in an MR environment is provided. The method of FIG. 6 represents a flowchart of a method 600 for performing, using wireless signal unit 60, 500, a dynamic adjustment of a transfer rate of wireless data to optimize the effective number of bits of the signal received at the unit, and thereby the data rate, to reduce power consumption and make the device and system more efficient. It should be understood that, unless clearly indicated to the contrary, in any methods claimed herein that include more than one step or act, the order of the steps or acts of the method is not necessarily limited to the order in which the steps or acts of the method are recited. The wireless unit 60, 500 and system may be any of those described or otherwise envisioned herein.

The wireless unit described herein can comprise a processor that is configured to determine a gradient signal activity value of a gradient system and dynamically adjust a transfer rate of wireless data transmitted from a transceiver of the unit based on the gradient signal activity value. In example embodiments, the processing hardware and/or software for determining the gradient signal activity can be located in the programmable resources within one or more of processors 64, 505, and/or 535. In alternate embodiments, the wireless transceiver can be configured to receive configuration information, data rate, etc., from any one or more of processors 64, 505, and/or 535 that are separate from the transceiver. In such embodiments, processors 64, 505, and/or 535 are configured to determine a gradient signal activity value of the gradient system and dynamically adjust a transfer rate of wireless data transmitted from the transceiver based on the gradient signal activity value. The wireless transceiver and/or the communication unit can receive the dynamically adjusted transfer rate from the one or more processors.

At step 610 of the method, a wireless monitoring system is provided within a healthcare facility. Providing the wireless monitoring system includes installing an MR system with an MR exam room having shielding walls made of electromagnetic interference (EMI) shielding material not limited to copper or steel in embodiments. Providing the wireless monitoring system further includes affixing one or more physiological sensors, such as the electrocardiography electrodes shown in FIGS. 1-3, to a patient in a suitable orientation to collect physiological data of the patient P. Providing the wireless monitoring system further includes connecting a wireless unit 60, 500 to the one or more physiological sensors and establishing a communication link CL between the wireless signal unit and a patient monitor 70. Both the wireless signal unit and the patient monitor are within the MR exam room for an MRI application. The patient monitor 70 is connected to the network of the healthcare facility.

At step 620 of the method, the patient is placed within a bore or an equivalent and an image acquisition process of an MR system is initiated. A patient support of the scanner can be positioned or adjusted, if necessary, prior to initiating the image acquisition process.

At step 630 of the method, magnetic resonance is generated and spatially encoded by selectively operating magnetic field gradient coils and RF coils, for example, in at least a portion of a region of interest of the patient.

At step 640 of the method, a signal corresponding to a physiological parameter of the patient is sensed by the one or more physiological sensors such as the ECG electrodes. The detected signal is received by a receiver coil or processor of the wireless signal unit. Transient gradient signals from the MR system are also sensed by the one or more physiological sensors.

At step 650 of the method, a processor of the wireless signal unit preprocesses the signal corresponding to the physiological parameter of the patient. The processor also determines a gradient signal activity value from the signal and/or the MR system. In embodiments, connection 74 is used to provide the data from the MR system. In other embodiments, the MR system data is obtained or otherwise accessed directly from controllers C or scanner 40. If the gradient signal activity value indicates that transient gradient signals are present, the transceiver of the wireless signal unit transmits the preprocessed wireless data associated with the input signal to the patient monitor at a default transfer rate at step 660 of the method. Such transmitted data is received by a receiver or transceiver of the patient monitor.

At step 670 of the method, the processor continues to receive and preprocess the signals corresponding to the physiological parameter of the patient during the image acquisition process. If a subsequent gradient signal activity value (or the initial gradient signal activity value for that matter) indicates the transient gradient signals are not present or less than a threshold value, the processor automatically adjusts or reduces the default data transfer rate and the transceiver 515 transmits the preprocessed wireless data at the adjusted or reduced transfer rate. Such transmitted data is received by a receiver or transceiver of the patient monitor. The processor can be configured to determine the effective number of bits of the received signal when the transient gradient signals are not present and compare the determined effective number of bits with a predetermined threshold or default effective number of bits used when the transient gradient signals are present. When the transient gradient signals are not present and the effective number of bits for the received signal is lower than the default number, the processor can reduce the transfer rate according to a rate or rate reduction that is associated with the lower effective number of bits. For example, a plurality of transfer rates can be stored in a lookup table in memory 510 where each transfer rate corresponds with an effective number of bits for the received signal. In another embodiment, a plurality of transfer rate reductions can be stored in a lookup table in memory 510 where each transfer rate reduction corresponds with an effective number of bits for the received signal relative to the default effective number of bits.

For example, the lookup table can include a default effective number of bits (ENOB) equal to 16, where the default ENOB is associated with a default transfer rate of X that represents a maximum or 100%. The lookup table can also include a first reduced effective number of bits that is equal to 15, where the first reduced ENOB is associated with a first transfer rate reduction expressed as a proportion of the default transfer rate. In such an embodiment, the first transfer rate reduction can be equal to 15/16 and the transfer rate can be adjusted to 93.75% of the maximum or default transfer rate. Similarly, the lookup table can include a second transfer rate reduction for a second reduced effective number of bits that is equal to 14. The second transfer rate reduction can be equal to 14/16 and the transfer rate adjustment associated with an ENOB of 14 can be 87.5% of the maximum or default transfer rate. The lookup table can include transfer rate values for each effective number of bits contemplated within the system so the processor doesn't need to calculate them repeatedly. In embodiments where the lookup table includes absolute transfer rate values, rather than proportional values, the lookup table can include a transfer rate of Y for the first reduced effective number of bits that is equal to 15 and another transfer rate of Z for the second reduced effective number of bits that is equal to 14 and so on. Embodiments with absolute values can be more appropriate in scenarios where the change in rate from 16 bits to 15 bits is different from the change in rate from 15 bits to 14 bits, for example.

At step 680 of the method, the patient monitor 70 of a patient information system receives the preprocessed data from the transceiver 515 at the default data transfer rate or the adjusted data rate. The patient monitor 70 can perform post-processing steps so that the data can be presented on display 72.

Wireless unit 60, 500 may use any number of wireless protocols including IEEE 802.11 protocols. The IEEE 802.11 standard is the family of specifications created by the Institute of Electrical and Electronics Engineers Inc. for wireless, local area networks in 2.4 and 5 gigahertz bandwidth spaces. It is a way to connect computers and other electronic devices to each other and/or to the Internet at high speeds without requiring wiring. Of course it should be appreciated that the present disclosure is not limited to any one particular wireless protocol. Any suitable protocol is contemplated including any suitable wireless communication technologies, such as, radio-frequency identification (RFID) technology, Wi-Fi technology, Bluetooth technology, technologies used by mobile phone communication systems such as cellular data technologies, and proprietary links, etc.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

1. A wireless signaler, comprising:
a transceiver configured to transmit, by a communication link, wireless data associated with a physiological parameter of a patient at a default data transfer rate within a patient information system; and
a processor configured to:
receive and preprocess an input signal comprising a signal corresponding to the physiological parameter of the patient and transient gradient signals from a gradient system;
determine a gradient signal activity value of the gradient system; and
dynamically adjust the default transfer rate of the wireless data transmitted from the transceiver to an adjusted data transfer rate based on the gradient signal activity value.

2. The wireless signaler of claim 1, wherein the gradient system is part of a magnetic resonance imaging (MRI) system.

3. The wireless signaler of claim 1, wherein the signal corresponding to the physiological parameter of the patient comprises an ECG signal.

4. The wireless signaler of claim 1, wherein the adjusted data transfer rate comprises a minimum rate needed to communicate the signal corresponding to the physiological parameter taking into consideration a data phase encoding process occurring during an image acquisition process.

5. The wireless signaler of claim 1, wherein the transceiver is configured to transmit the wireless data associated with the input signal to a patient monitor comprising a display, and wherein the patient monitor is part of the patient information system or otherwise connected to the patient information system.

6. The wireless signaler of claim 1, wherein the processor is further configured to determine an effective number of bits of the received input signal.

7. The wireless signaler of claim 6, wherein the processor is further configured to compare the determined effective number of bits of the received input signal with a predetermined or default maximum effective number of bits used when the transient gradient signals are present.

8. The wireless signaler of claim 7, wherein the processor is further configured to reduce the default data transfer rate when the determined effective number of bits of the received input signal is lower than the predetermined or default maximum effective number of bits used when the transient gradient signals are present.

9. The wireless signaler of claim 8, wherein the processor is further configured to reduce the default transfer rate according to a predetermined reduced transfer rate associated with the determined effective number of bits of the received input signal.

10. A system for monitoring a physiological parameter of a patient, comprising:
a wireless signaler comprising a transceiver configured to transmit, by a communication link, wireless data associated with the physiological parameter of the patient at a default data transfer rate;
a patient monitor comprising a receiver configured to receive the wireless data transmitted from the transceiver of the wireless transceiver at the default data transfer rate; and
a processor communicably coupled with the wireless signaler, wherein the processor is configured to: (i) receive and preprocess an input signal comprising a signal corresponding to the physiological parameter of the patient and transient gradient signals from a gradient system; (ii) determine a gradient signal activity value of the gradient system; and (iii) dynamically adjust the default data transfer rate of the wireless data transmitted from the transceiver to an adjusted transfer rate based on the gradient signal activity value.

11. The system of claim 10, wherein the gradient system is part of a magnetic resonance imaging (MRI) system.

12. The system of claim 10, wherein the signal corresponding to the physiological parameter of the patient comprises an ECG signal.

13. The system of claim 10, wherein the adjusted data transfer rate comprises a minimum rate needed to communicate the signal corresponding to the physiological parameter taking into consideration a data phase encoding process occurring during an image acquisition process.

14. The system of claim 10, wherein the patient monitor further comprises:
a monitor processor configured to post-process the wireless data received from the transceiver; and
a display configured to display the post-processed wireless data.

15. The system of claim 10, wherein the processor is further configured to: (i) determine an effective number of bits of the received input signal; (ii) compare the determined effective number of bits of the received input signal with a predetermined or default maximum effective number of bits used when the transient gradient signals are present; and (iii) reduce the default data transfer rate when the determined effective number of bits of the received input signal is lower than the predetermined or default maximum effective number of bits used when the transient gradient signals are present.
